(19) United States
(12) Patent Application Publication
(10) Pub. No.: US 2019/0254979 A1
(43) Pub. Date: Aug. 22, 2019

(54) SOFT PLANT-BASED CAPSULE COMPOSITION HAVING IMPROVED ECONOMIC FEASIBILITY AND PRODUCTIVITY BY MINIMIZING QUALITY VARIATION

(71) Applicant: CHANGSUNGSOFTGEL CO.,LTD, Pocheon-si (KR)

(72) Inventor: Ju-Su KIM, Seoul (KR)

(21) Appl. No.: 16/401,668

(22) Filed: May 2, 2019

Related U.S. Application Data
(63) Continuation of application No. PCT/KR2018/008821, filed on Aug. 3, 2018.

(30) Foreign Application Priority Data
Sep. 28, 2017 (KR) 10-2017-0126511

(51) Int. Cl. A61K 9/48; A61K 47/02; A61K 47/10; A61K 47/36; A23P 10/30; A23L 29/219; A23L 29/256; A23L 29/00

(52) U.S. Cl. CPC A61K 9/4816; A61K 47/02; A61K 47/10; A61K 47/36; A23V 2002/00; A23L 29/219; A23L 29/256; A23L 29/015; A23L 29/035; A23P 10/30

(57) ABSTRACT

A vegetable capsule composition is provided. More particularly, in a state of using only one type of starch and treating the starch with hydroxypropyl and acid to have improved viscosity stability, water retention and transparency, a soft vegetable capsule is formed by mixing iota carrageenan having high viscosity and glucose, so as to easily manage the starch while minimizing the quality deviation, and to universally use the capsule to improve a condition of providing capsule molding equipment, and thus the improved economic efficiency and productivity are implemented.

### CROSS-REFERENCE TO RELATED APPLICATION(S)

This application is a continuation application, claiming priority under § 365(c), of an International application No. PCT/KR2018/008821, filed on Aug. 3, 2018, which was based on and claimed the benefit of a Korean patent application number 10-2017-0126511, filed on Sep. 28, 2017, in the Korean Intellectual Property Office, the disclosure of which is incorporated by reference herein in its entirety.

### BACKGROUND

### 1. Field

The disclosure relates to a soft vegetable capsule composition. More particularly, the disclosure relates to a soft vegetable capsule composition for easily managing starch while minimizing quality variation, and improving a condition of capsule molding equipment by using an animal molding machine and a spreader box in combination without using an additional vegetable molding machine, a slot die, a metering station and a cooling conveyor, so that improved economy and productivity are implemented.

### 2. Description of the Related Art

In general, a tablet and a capsule are used to administer an accurate amount of food or medicine (hereinafter referred to as “contents”) to be ingested. In particular, the capsule is excellent in shielding the taste and odor due to the contents to be ingested.

However, gelatin used as a main raw material for the capsule is a kind of derived protein obtained by transforming collagen, which is insoluble protein contained in a skin, bone, tendon or the like of a cow or pig, into water soluble state by decomposing and refining the collagen after heated with water. Thus, the gelatin has little taste and smell, and remains in a semi-solid (gel) state at room temperature.

However, recently, there is a trend to avoid using animal gelatin due to aversion and side effects such as mad cow disease and allergy.

Accordingly, vegetable capsules using starch and carrageenan as vegetable materials instead of animal materials have been used recently.

However, most of the vegetable capsules have a thick coating film, low elasticity, and difficulty to dissolve.

The applicant of the disclosure has disclosed a soft vegetable capsule composition including 35.3 wt %±10 wt % of starch and carrageenan, 17.7 wt %±3 wt % of glycerin, and 47 wt %±7 wt % of purified water, wherein

the starch and carrageenan (35.3 wt %) are composed of 24.71 wt %±1.7 wt % and 10.59 wt %±1.7 wt %, respectively;

the starch is composed of 17.65 wt %±2 wt % of cornstarch, 3.53 wt %±1 wt % of tapioca starch, and 3.53 wt %±1 wt % of potato starch; and

the carrageenan is composed of 7.592 wt %±1 wt % of iota carrageenan, 2.118 wt %±0.641 wt % of kappa carrageenan, 0.8 wt %±0.3 wt % of sodium bicarbonate (sodium hydrogen carbonate), 0.04 wt %±0.03 wt % of Arabia gum, and 0.04 wt %±0.03 wt % of levan (see Korean Patent Registration No. 10-1467853).

However, according to the above soft vegetable capsule composition, because three different types of cornstarch, tapioca starch and potato starch are mixed at different ratios, quality deviation inevitably occurs when used.

In particular, since the starch characteristics are different from each other, each starch is required to be separately managed according to a standard.

In addition, it is difficult to use a spreader box because the gelatin has low fluidity, and the metering station, slot die, and cooling conveyor are additionally required because the different starches are mixed with the carrageenan for use.

(Patent Document 1) KR10-1467853 B1

(Patent Document 2) KR10-1359469 B1

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a vegetable capsule composition configured to easily manage starch while minimizing quality deviation when the vegetable capsule is manufactured, and be prepared even when a conventional animal molding machine and a spreader box are used in combination without using an additional vegetable molding machine, a slot die, a metering station and a cooling conveyor.

Additional aspects will be set forth in part in the description which follows and, in part, will be apparent from the description, or may be learned by practice of the presented embodiments.

To achieve the above-mentioned object, the vegetable capsule composition according to the disclosure is obtained by mixing iota carrageenan, which has high viscosity to easily form starch into a film, with glucose facilitating a network structure of the iota carrageenan, in a state of using only one kind of starch and treating the starch with hydroxypropyl and acid to have improved viscosity stability, water retention and transparency.

According to the disclosure, only one kind of cornstarch is used, so that costs can be lowered and quality deviation can be minimized.

Further, according to the disclosure, only one kind of cornstarch is managed, so that the management can be easily performed.

In addition, according to the disclosure, adhesiveness and transparency are improved as compared with the conventional vegetable capsules, and the adhesiveness is maintained even when amounts of glycerin and purified water are increased to secure fluidity, so that

the vegetable capsule composition can be prepared through viscosity adjustment by using the conventional animal molding machine and the spreader box even without using the vegetable molding machine, metering station, slot die and cooling conveyor, and thus a condition of providing the capsule molding equipment can be improved, and economic efficiency and productivity can be improved.

Other aspects, advantages, and salient features of the disclosure will become apparent to those skilled in the art from the following detailed description, which, taken in conjunction with the annexed drawings, discloses various embodiments of the disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Hereinafter, the disclosure will be described in detail based on examples.

First, the vegetable capsule composition of the disclosure is obtained and used by mixing iota carrageenan, which has high viscosity to easily form a film, with glucose facilitating a network structure of the iota carrageenan, in a state of using only one kind of starch and treating the starch with hydroxypropyl and acid to have improved viscosity stability, water retention and transparency.

TABLE 1

| | | Raw material | | |
|---|---|---|---|---|
| No. | Class. | Mixed ratio (%) | Composed ratio (%) | |
| 1 | Starch | 28.8% ± 12% | 71.2% | Cornstarch |
| 2 | Carrageenan | | 28.8% | Iota carrageenan |
| | | | | (85-92%) |
| | | | | Glucose (8-15%) |

| | | | |
|---|---|---|---|
| 3 | Buffer | 2% ± 1.5% | Sodium polyphosphate |
| 4 | Glycerin | 20.7% ± 7% | 20.7% |
| 5 | Purified | 48.5% ± 5% | 48.5% |
| | water | | |

| | | |
|---|---|---|
| Total | 100% | 100% |

Herein, as shown in the above table, the gelatin composition for preparing the capsule is composed of starch and carrageenan, buffer, glycerin, and purified water, and the mixed ratio is as follows.

### Example 1

Starch and Carrageenan: 28.8%±12%

Buffer: 2%±1.5%

Glycerin: 20.7%±7%

Purified water: 48.5%±5%

In the above case, the starch and carrageenan occupy 28.8%±12% (16.8% to 40.8%) based on the total mixed ratio.

When the starch and carrageenan are less than 16.8%, which is out of the error range, the viscosity is lowered when the contents are accommodated, and thus the water retention and stability are deteriorated.

When the starch and carrageenan are more than 40.8%, which is out of the error range, the starch and carrageenan tend to have a hardness after a predetermined time, and cracks may occur according to some examples.

The composed ratio of the starch and carrageenan based on the total mixed ratio for the starch and carrageenan is as follows.

### Example 2

Starch: 71.2%

Carrageenan: 28.8%

In the above case, the starch and carrageenan are maintained at the ratio of about 7:3. In particular, only one kind of starch is used as 100% of the starch, so that the management is easily performed while the quality deviation is minimized.

According to the disclosure, cornstarch, which has low price and fine particles, is used for the above starch.

In particular, the starch becomes gelatinized and has viscosity when heated after water is added, thereby improving stability and adhesiveness.

In addition, when the starch is heated, clear and transparent light is expressed, so that transparency is improved.

Therefore, the cornstarch is treated with hydroxypropyl and acid to have improved viscosity stability, water retention and transparency, and then the cornstarch treated as above is mixed with the carrageenan for use.

In addition, the composed ratio of the carrageenan (28.8%) mixed with the cornstarch (71.2%) is shown as follows.

### Example 3

Iota carrageenan: 85% to 92%

D-glucose (Dextrose monohydrate): 8% to 15%

In the above case, the carrageenan is a material derived from seaweed (Chondrus crispus), and has a copolymer structure of potassium, sodium, magnesium, calcium and ammonium sulfate ester of 3,6-anhydrous-D-galactose having α-1,3 or β-1,4 bonded hexose and D-galactose, in which the carrageenan is divided into iota carrageenan, kappa carrageenan and lambda carrageenan depending on the position and number of sulfate groups of the hexose structure, and only the iota carrageenan is used in the disclosure.

Herein, the iota carrageenan has a sulfate group at position 2 of the hexose, and has the high viscosity and purity, so that gel having high density is formed.

In other words, the iota carrageenan has different gelation characteristics depending on purity and distribution of molecular weight, in which the iota carrageenan having high purity or high viscosity forms a firm gel even at low concentration. Typically, the iota carrageenan may be 85% or more as a polymer type having an average density of 50,000 DT or more.

In addition, the D-glucose (Dextrose monohydrate), which is glucose, is used adjust viscosity and jelly strength to facilitate the formation of network structures of the iota carrageenan and the cornstarch.

In addition, the buffer is formed of a metal salt such as calcium lactate, potassium metaphosphate and calcium oxide. According to the disclosure, about 0.5% to 3.5% of sodium polyphosphate may be used based on the total mixed ratio.

In addition, the glycerin is used as a plasticizer for moldability, and the glycerin may be used within a range of 20.7%±7% (13.7% to 27.7%) based on the total mixed ratio.

When the glycerin is less than 13.7%, which is out of the error range, the capsule hardness increases, thereby cutting a gelatin ribbon and lowering the adhesiveness, so that defects may be caused.

When the glycerin is more than 27.7%, which is out of the error range, the hardness is decreased and the elasticity is deteriorated, so that the ribbon is rarely formed, and thus a finished product may be broken.

In addition, the purified water serves as a solvent used for mixing the glycerin, starch, carrageenan and buffer, and is added in the range of 48.5%±5% (43.5% to 53.5%) based on the total mixed ratio.

When the purified water is more than 53.5%, which is out of the error range, the density of a film is lowered to prevent the film from being formed. When the purified water is less than 43.5%, which is out of the error range, the mixing is insufficiently conducted, thereby increasing the hardness, and thus the film may not be formed.

Therefore, according to the disclosure, only one kind of cornstarch is mixed and used at a predetermined ratio, so that the cost can be lowered and the quality deviation can be minimized. Only one kind of cornstarch is managed, so that the management is easily performed.

In addition, according to the disclosure, the adhesiveness and transparency are improved by using only one kind of cornstarch, and the adhesiveness is maintained even when amounts of the glycerin and the purified water are increased to secure fluidity.

Therefore, the capsule is prepared through viscosity adjustment by using the conventional animal molding machine and the spreader box even without using the vegetable molding machine, metering station, slot die and cooling conveyor, so that the economic efficiency and productivity can be improved.

While the disclosure has been shown and described with reference to various embodiments thereof, it will be understood by those skilled in the art that various changes in form and details may be made therein without departing from the spirit and scope of the disclosure as defined by the appended claims and their equivalents

What is claimed is:

1. A vegetable capsule composition comprising:
starch, carrageenan, buffer, glycerin, and purified water,
wherein the starch is cornstarch treated with hydroxypropyl and acid,
wherein the carrageenan includes 85 wt % to 92 wt % of iota carrageenan formed in a polymer type having an average density of 50,000 DT or more, and 8 wt % to 15 wt % of D-glucose (Dextrose monohydrate) as glucose,
ratios of the starch and the carrageenan are 71.2 wt % and 28.8 wt %, respectively, in which a sum of the starch and the carrageenan corresponds to 28.8 wt %±12 wt % based on 100 wt % as a total weight,
sodium polyphosphate used as the buffer corresponds to 2 wt %±1.5 wt % based on the total weight,
glycerin corresponds to 20.7 wt %±7 wt % based on the total weight, and
the purified water corresponds to 48.5 wt %±5 wt % based on the total weight.
